(19) United States
(12) Patent Application Publication
(10) Pub. No.: US 2026/0069523 A1
(43) Pub. Date: Mar. 12, 2026

(54) SKIN CARE COMPOSITION AND USE THEREOF

(71) Applicant: NANJING NUTRABUILDING BIO-TECH CO., LTD., Nanjing (CN)

(72) Inventors: Ronghua YI, Nanjing (CN); Qianjing ZHANG, Nanjing (CN); Kylin LIAO, Nanjing (CN); Shawn WELLS, Frisco, TX (US)

(21) Appl. No.: 19/391,491

(22) Filed: Nov. 17, 2025

Related U.S. Application Data
(63) Continuation of application No. PCT/CN2024/093241, filed on May 15, 2024.

(30) Foreign Application Priority Data
May 18, 2023 (WO) PCT/CN2023/095031

(51) Int. Cl. A61K 8/49; A61K 8/34; A61K 8/35; A61K 8/60; A61K 8/63; A61K 8/64; A61K 8/67; A61K 8/68; A61K 8/73; A61K 8/9789; A61K 8/9794; A61Q 19/00; A61Q 19/02; A61Q 19/08

(52) U.S. Cl. CPC A61K 8/4953; A61K 8/347; A61K 8/35; A61K 8/4906; A61K 8/498; A61K 8/602; A61K 8/63; A61K 8/64; A61K 8/671; A61K 8/676; A61K 8/68; A61K 8/731; A61K 8/9789; A61K 8/9794; A61Q 19/007; A61Q 19/02; A61Q 19/08

(57) ABSTRACT

The present invention discloses a skin care composition, comprising a first active component and a second auxiliary component. The first active component is selected from one or more of tetrahydrocurcumin, ergothioneine, phloretin, arbutin, resveratrol, vitamin C, dipotassium glycyrrhizinate, ceramide, Aloe vera polysaccharide, Opuntia dillenii polysaccharide, Astragali radix polysaccharide, Dendrobii caulis polysaccharide, Cucumis metuliferus polysaccharide, fucoidan, small-molecule hyaluronic acid, pearl powder, collagen, tea tree essential oil, thearubigins, salicylic acid, retinol, glycoxyl, carnosine; the second auxiliary component is selected from one or more of tetrahydropiperine, laurocapram, PEG-75 shea butter glycerides, hydrogenated castor oil, glycerol, lauric acid, sucrose laurate, butanediol, polysorbate, hydroxyethyl cellulose, 1,2-pentanediol, ethoxydiglycol. The skin care composition of the present invention can be used for whitening, anti-inflammatory, anti-glycation, moisturizing, skin firming, anti-bacterial and/or anti-aging.

### CROSS REFERENCE TO RELATED APPLICATION

This application is a continuation application of International Patent Application No. PCT/CN2024/093241, filed on May 15, 2024, which claims the priority of the International Patent Application No. PCT/CN2023/095031, filed on May 18, 2023, the contents of all of which are incorporated herein by reference in their entirety.

### FIELD OF THE INVENTION

The invention belongs to the technical field of cosmetics, and in particular relates to a skin care composition which can be used for whitening, anti-inflammatory, anti-glycation, moisturizing, skin firming, anti-bacterial and/or anti-aging.

### BACKGROUND OF THE INVENTION

With the rapid development of society, the problem of environmental pollution has become increasingly prominent. In a polluted environment for a long time, the skin is vulnerable. In addition, the pressure faced by human beings in the fast-paced life is increasing; coupled with the influence of ultraviolet, age, drugs and other factors, the skin is prone to various problems, which will affect the appearance and even cause skin health problems.

With the continuous development of science and technology and economy, the pursuit of beauty and health has promoted the continuous and in-depth research in the field of skin care. People are increasingly interested in skin care compositions and cosmetics that can achieve and improve skin whitening, anti-inflammatory, anti-glycation, moisturizing, skin firming, anti-bacterial and anti-aging effects.

### SUMMARY OF THE INVENTION

In one aspect, the present invention provides a skin care composition, comprising a first active component and a second auxiliary component.

In some embodiments, the skin care is one or more of relieving photoaging of skin, reducing fine lines and wrinkles of skin, improving skin elasticity, improving skin compactness, improving skin gloss, brightness or texture, reducing or eliminating acne, relieving skin inflammation, keeping skin moist, improving or evening skin tone. In some embodiments, the skin can be any skin surface selected from scalp, face, neck, shoulders, back, arms, legs, hands, feet, chest, abdomen and buttocks.

In some embodiments, the first active component is selected from one or more of tetrahydrocurcumin, ergothioneine, phloretin, arbutin, resveratrol, vitamin C, dipotassium glycyrrhizinate, ceramide, Aloe vera polysaccharide, Opuntia dillenii polysaccharide, Astragali radix polysaccharide, Dendrobii caulis polysaccharide, Cucumis metuliferus polysaccharide, fucoidan, small-molecule hyaluronic acid, pearl powder, collagen, tea tree essential oil, thearubigins, salicylic acid, retinol, glycoxyl, carnosine; the second auxiliary component is selected from one or more of tetrahydropiperine, laurocapram, PEG-75 shea butter glycerides, hydrogenated castor oil, glycerol, lauric acid, sucrose laurate, butanediol, polysorbate, hydroxyethyl cellulose, 1,2-pentanediol, ethoxydiglycol. In some embodiments, the first active component can be any suitable active component that can exert whitening, anti-inflammatory, anti-glycation, moisturizing, skin firming, anti-bacterial, anti-aging and other effects; the second auxiliary component can be any suitable auxiliary component that can cooperate with the first active component and/or promote the effect of the first active component. In some embodiments, retinol can be replaced by other retinoids selected from tretinoin, isotretinoin, alitretinoin, acitretin, etretinate, adapalene, tazarotene, and derivative thereof.

In some embodiments, the weight ratio of the first active component to the second auxiliary component is 1:20 to 50:1. In some embodiments, the weight ratio of the first active component to the second auxiliary component may be 1:50 to 100:1, 1:10 to 20:1.

In some embodiments, the first active component is selected from one or more of tetrahydrocurcumin, ergothioneine, phloretin, arbutin, resveratrol, vitamin C, dipotassium glycyrrhizinate, ceramide, Opuntia dillenii polysaccharide, Astragali radix polysaccharide, Dendrobii caulis polysaccharide, Cucumis metuliferus polysaccharide, thearubigins, retinol, carnosine.

In some embodiments, the first active component is selected from one or more of tetrahydrocurcumin, ergothioneine.

In some embodiments, the second auxiliary component is selected from one or more of tetrahydropiperine, laurocapram, hydrogenated castor oil, glycerol, sucrose laurate, butanediol, 1,2-pentanediol. In some embodiments, the second auxiliary component is selected from one or more of tetrahydropiperine, laurocapram, sucrose laurate.

In some embodiments, the second auxiliary component is selected from one or more of tetrahydropiperine, laurocapram.

In some embodiments, the first active component is tetrahydrocurcumin and/or ergothioneine, the second auxiliary component is tetrahydropiperine.

In some embodiments, the weight ratio of the first active component to the second auxiliary component is 1:2 to 5:1. In some embodiments, the weight ratio of the first active component to the second auxiliary component may be 1:5 to 10:1, 1:1 to 2:1.

In some embodiments, the composition of the invention inhibits tyrosinase activity by about 80%, preferably by about 85% and above 90%.

In some embodiments, the composition of the invention can inhibit TNF-α, IL-6 or IL-8 by more than 20% respectively. Preferably, it can inhibit TNF-α secretion by about 25%, IL-6 secretion by about 40% and IL-8 secretion by about 55%.

In some embodiments, the composition of the invention can inhibit AGEs by about 50%.

In some embodiments, the skin care composition is used for whitening, anti-inflammatory, anti-glycation, moisturizing or skin firming. In some embodiments, the skin care composition can be used for whitening, anti-inflammatory, anti-glycation, moisturizing, skin firming, anti-bacterial, anti-aging and other effects.

In another aspect, the present invention provides use of the skin care composition herein in the preparation of a cosmetic.

In another aspect, the present invention provides a cosmetic comprising the skin care composition as described herein.

In some embodiments, the cosmetic includes lotion, essence, powder, emulsion, cream, gel, mask and dressing.

In some embodiments, the weight ratio of the skin care composition in the cosmetic is 0.1-95%. In some embodiments, the weight ratio of the skin care composition in the cosmetic may be 1-90%, 2-80%, 5-70%, 10-60%.

In some embodiments, the weight ratio of the skin care composition in the cosmetic is 20-50%.

The composition of the present invention can make all effective ingredients and/or active components cooperate with each other to play a greater role synergistically, and can be widely used in various skin care products and cosmetics.

### BRIEF DESCRIPTION OF THE FIGURES

FIG. 1 shows the cytotoxicity of THC, EGT and THP at different doses (1 μM, 10 μM, 50 μM and 100 μM).

FIG. 2 shows effects of THC, THC+THP, EGT, EGT+THP on inflammatory factor TNF-α.

FIG. 3 shows effects of THC, THC+THP, EGT, EGT+THP on inflammatory factor IL-6.

FIG. 4 shows effects of THC, THC+THP, EGT, EGT+THP on inflammatory factor IL-8.

FIG. 5 shows inhibitory effects of THC, EGT, THC+THP, EGT+THP and THC+EGT+THP on tyrosinase activity.

FIG. 6 shows the inhibition rate of the composition of Example 12 on AGEs.

FIG. 7 shows the colonization of Propionibacterium acnes.

### DETAILED DESCRIPTION OF THE INVENTION

Reference will now be made in detail to the preferred embodiments of the invention, examples of which are further illustrated. While the invention will be described in conjunction with the preferred embodiments, it will be understood that they are not intended to limit the invention to these embodiments. To the contrary, the invention is intended to cover alternatives, modifications and equivalents, which may be included within the spirit and scope of the invention as defined by the claims. Furthermore, in the detailed description of the present invention, numerous specific details are set forth in order to provide a thorough understanding of the present invention. However, it will be obvious to one of ordinary skill in the art that the present invention may be practiced without these specific details. In other instances, well known methods, procedures, components, and other features have not been described in detail as not to unnecessarily obscure aspects of the present invention.

As used herein, the term “or” is meant to include both “and” and “or.” In other words, the term “or” may also be replaced with “and/or.”

As used herein, the singular forms “a,” “an” and “the” are intended to include the plural forms as well, unless the context clearly indicates otherwise.

As used herein, the term “comprise” or “include” or their conjugations, refer to a situation where said terms are used in their non-limiting sense to mean that items following the word are included, but items not specifically mentioned are not excluded. It also encompasses the more limiting verb ‘to consist essentially of’ and ‘to consist of’.

A plurality of skin care ingredients are used in the composition of the invention, which can work together to achieve a truly effective anti-skin problem effect.

Tetrahydropiperine, which is extracted from plants of Piperaceae, is a safe and efficient natural plant-extracted osmolyte. By promoting the flow of phospholipid bilayer in cells, it drives functional active components to penetrate into cells. Components that have similar penetration enhancer or adjuvant effect to tetrahydropiperine and can be used in cosmetics include but are not limited to: laurocapram, PEG-75 shea butter glycerides, hydrogenated castor oil, glycerol, lauric acid, sucrose laurate, butanediol, polysorbate, hydroxyethyl cellulose, 1,2-pentanediol, ethoxydiglycol, etc.

The skin care composition of the present invention is simple and convenient to prepare, and can be prepared by uniformly mixing all the raw materials. In some embodiments, the preparation method of the skin care composition of the present invention includes the following steps: firstly, uniformly mixing the raw materials with low content, and then mixing them with the raw materials with high content in equal increments. More preferably, the raw materials are mixed in a three-dimensional way for 20-30 minutes. In some embodiments, various components in the skin care composition of the present invention can each be dissolved in a suitable solvent to form a corresponding solution and/or suspension respectively, and then the solutions and/or suspensions of the various components are mixed to form a mixed form of the skin care composition of the present invention dissolved in a solvent. In the preparation of the skin care composition, various components can be reasonably added according to the properties of the raw materials by using a mixing method well known in the art.

The technical features, implementation methods and beneficial effects of the invention will be further described in detail with specific examples. The following examples are only part of the embodiments of the present invention, but not all of them. Unless otherwise specified, the materials and reagents described in the following examples are all common commercial products and can be purchased in the market.

### EXAMPLES

### Example 1

The composition of Example 1 was prepared by mixing ergothioneine, tetrahydropiperine and sucrose laurate in a weight ratio of 1:3:2.

### Example 2

The composition of Example 2 was prepared by mixing tetrahydrocurcumin, tetrahydropiperine and hydroxyethyl cellulose with the same weight.

### Example 3

The composition of Example 3 was prepared by mixing tetrahydrocurcumin, ergothioneine, tetrahydropiperine, laurocapram and sucrose laurate in a weight ratio of 2:2:3:1:2.

### Example 4

The composition of Example 4 was prepared by mixing tetrahydrocurcumin and tetrahydropiperine with the same weight.

### Example 5

The composition of Example 5 was prepared by mixing ergothioneine and tetrahydropiperine with the same weight.

### Example 6

The composition of Example 6 was prepared by mixing ergothioneine, arbutin, phloretin, tetrahydropiperine, sucrose laurate in a weight ratio of 3:2:1:3:1.

### Example 7

The composition of Example 7 was prepared by mixing tetrahydrocurcumin, ergothioneine, resveratrol, glycerol, laurocapram in a weight ratio of 2:2:1:1:2.

### Example 8

The composition of Example 8 was prepared by mixing tetrahydrocurcumin, ergothioneine and tetrahydropiperine with the same weight.

### Example 9

The composition of Example 9 was prepared by mixing vitamin C, dipotassium glycyrrhizinate, phloretin, ceramide, tetrahydropiperine, sucrose laurate with the same weight.

### Example 10

The composition of Example 10 was prepared by mixing resveratrol, pearl powder, fucoidan, arbutin, tetrahydropiperine in a weight ratio of 1:2:1:1:2.

### Example 11

The composition of Example 11 was prepared by mixing tetrahydrocurcumin, retinol, arbutin, dipotassium glycyrrhizinate, Opuntia dillenii polysaccharide, tetrahydropiperine, sucrose laurate and hydroxyethyl cellulose in a weight ratio of 3:2:2:1:2:1:1:1.

### Example 12

The composition of Example 12 was prepared by mixing ergothioneine, vitamin C, resveratrol, Aloe vera polysaccharide, tetrahydropiperine, hydroxyethyl cellulose in a weight ratio of 3:3:3:1:1:1.

### Example 13

The composition of Example 13 was prepared by mixing pearl powder, carnosine, Aloe vera polysaccharide, hydroxyethyl cellulose in a weight ratio of 4:3:1:1.

### Example 14

The composition of Example 14 was prepared by mixing tetrahydrocurcumin, ergothioneine, carnosine, ceramide, arbutin, laurocapram and polysorbate in a weight ratio of 3:2:1:3:1:1:1.

### COMPARATIVE EXAMPLE

Comparative examples include compositions comprising only the first active ingredient, and existing skin care compositions.

### Property Tests

Anti-inflammatory test: The anti-inflammatory effect of the composition of the present invention on skin keratinocytes was tested.

The reagents used include: DMED medium, fetal calf serum (FBS), penicillin/streptomycin, MTT cell activity detection kit, cholecalciferol, dimethyl sulfoxide (DMSO), TNF-α kit, IL-8 kit, IL-6 kit, tetrahydrocurcumin (THC), ergothioneine (EGT), tetrahydropiperine (THP), and the compositions of Examples 1-14 of the present invention. Instruments include: cell incubator, cell counter, microplate reader.

Cell culture: HaCat, a normal human epidermal keratinocyte (NHEK) cell line, was purchased from Wuhan Saios Biotechnology Co., Ltd. The cells were stored in a humid environment (95% air and 5% carbon dioxide at 37° C.) and cultured in DMEM supplemented with 10% heat inactivated FBS, 2 mM glutamine and 1% penicillin/streptomycin. The culture was harvested and the cell morphology was examined by phase contrast microscope. Cell suspension was counted with a hemocytometer (Marienfeld, Germany) to monitor the number of cells.

Cytotoxicity test: MTT method was used to evaluate the effect of the composition of the invention on the vitality of normal human epidermal keratinocyte cell line. Briefly, cells were inoculated into a 96-well plate, treated with the composition of the example of the present invention with concentrations of 1 μM, 10 μM, 50 μM and 100 μM for 72 h, and incubated with MTT solution (100 μg/well) for 4 h. Formazine precipitate was dissolved in dimethyl sulfoxide, and the absorbance at 570 nm was determined by a microplate reader, and the reduction degree of MTT was quantitatively determined.

UVB stimulation: Keratinocytes were inoculated into a 12-well plate (50,000 cells/mL) and cultured to a confluency of ˜80%. The cells were washed once with phosphate buffered saline (PBS) at room temperature, 0.5 mL PBS was added to the cells, and then irradiated with ultraviolet rays. The cells were exposed to 50 mJ/cm² UV-B (Philips, 311 nm) for 2 minutes. Immediately after irradiation, PBS was replaced with media containing THC (maximum non-cytotoxic dose), EGT (maximum non-cytotoxic dose), THC (maximum non-cytotoxic dose/2)+THP (maximum non-cytotoxic dose/2), EGT (maximum non-cytotoxic dose/2)+THP (maximum non-cytotoxic dose/2) or control (DMSO), respectively. The cells were stored at 37° C. for 24 h. Cell culture supernatant was collected and stored at −80° C. for use.

Detection of inflammatory factors: the standard curve of each cytokine (TNF-α, IL-8, IL-6) were generated by using the reference cytokine samples provided by the kit. The cell culture supernatant was pre-coated with antibodies at the bottom of the microplate, incubated and thoroughly washed. Using substrate TMB to develop color, TMB turned blue under the catalysis of peroxidase and finally yellow under the action of acid. The color depth is positively correlated with TNF-α, IL-8 and IL-6 in the sample. The absorbance (OD value) was measured by microplate reader at the wavelength of 450 nm, and the concentration was calculated according to the standard curve.

Preparation of tetrahydrocurcumin+tetrahydropiperine test solution: 1 mg/mL THC solution and 1 mg/mL THP solution were mixed at 1:1. Preparation of ergothioneine+tetrahydropiperine test solution: 1 mg/mL EGT solution and 1 mg/ml THP solution were mixed at 1:1.

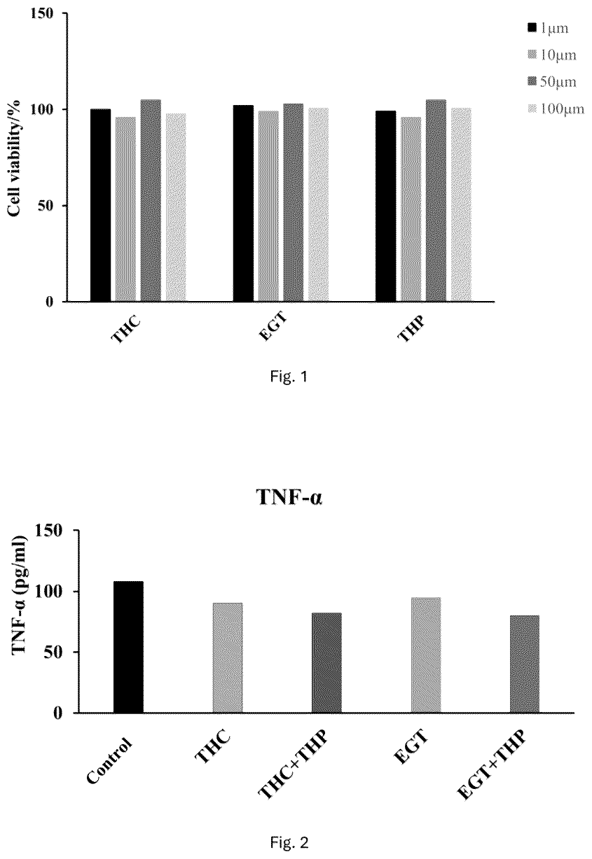

FIG. 1 shows the cytotoxicity of THC, EGT and THP at different doses (1 μM, 10 μM, 50 μM and 100 μM). As can be seen from FIG. 1, several doses of substances are not toxic to cells, and the cell activity is about 100% and not less than 85%.

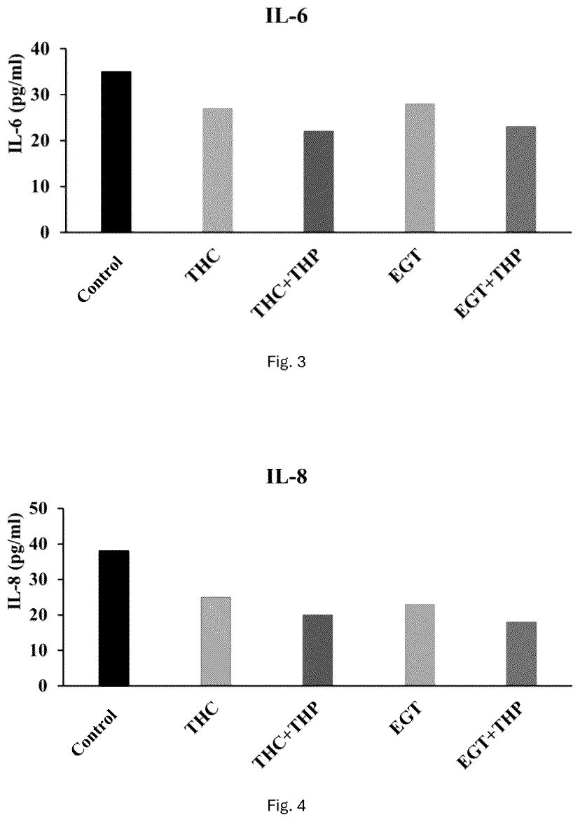

FIG. 2, FIG. 3 and FIG. 4 respectively show the anti-inflammatory effects of THC, THC+THP, EGT, EGT+THP on ultraviolet damaged cells. As shown in FIG. 2, the content of TNF-α is about 90 pg/ml under THC treatment, and it is about 82 pg/ml when THC+THP is used together. The content of TNF-α under EGT treatment is about 95 pg/ml, and it is about 80 pg/ml when EGT+THP is used together. The above data show that the combination group has a good inhibitory effect on inflammatory factor TNF-α. As shown in FIG. 3, the content of IL-6 under THC treatment is 27 pg/ml, and it is 22 pg/ml when THC+THP is used together. The content of IL-6 under EGT treatment is 28 pg/ml, and it is 23 pg/ml when EGT+THP is used together. The above data show that the combination group has a good inhibitory effect on inflammatory factor IL-6. As shown in FIG. 4, the content of IL-8 under THC treatment is 25 pg/ml, and it is 20 pg/ml when THC+THP is used together. The content of IL-8 under EGT treatment is 23 pg/ml, and it is 18 pg/ml when EGT+THP is used together. The above data show that the combination group has a good inhibitory effect on inflammatory factor IL-8. As can be seen from FIG. 2, FIG. 3 and FIG. 4, THC, THC+THP, EGT and EGT+THP can inhibit the production of inflammatory factors TNF-α, IL-6 and IL-8. THC and EGT have anti-inflammatory effects on ultraviolet damage of cells, and their anti-inflammatory effects can be significantly enhanced when combined with THP.

The results show that the composition of the present invention has significantly enhanced anti-inflammatory effect on ultraviolet damage of cells. Compared with the inflammatory factors secreted by the control group, the composition of the invention inhibits TNF-α secretion by about 25%, IL-6 secretion by about 40% and IL-8 secretion by about 55%.

Whitening test: The inhibitory effect of the composition of the present invention on tyrosinase was tested.

The reagents used include tyrosinase, tyrosine, potassium chloride, sodium chloride, potassium dihydrogen phosphate, disodium hydrogen phosphate, tetrahydrocurcumin (THC), ergothioneine (EGT), tetrahydropiperine (THP), deionized water and dimethyl sulfoxide (DMSO). The instruments include: magnetic stirrer, microplate reader.

Reagent preparation: phosphate buffer solution (PBS): 0.2 g potassium chloride, 8 g sodium chloride, 0.27 g potassium dihydrogen phosphate and 3.58 g disodium hydrogen phosphate were added to 800 mL distilled water, and stirred with a magnetic stirrer until completely dissolved, then the pH value was adjusted to 6.8, and the volume was adjusted to 1000 mL. After autoclaving, it can be stored at 4° C. or at room temperature. 2 mM L-tyrosine solution: 36 mg of L-tyrosine was weighed to 100 mL of water, stirred by a magnetic stirrer until it was completely dissolved to obtain the L-tyrosine solution. Preparation of tetrahydrocurcumin test solution: 10 mg of THC was dissolved in 10 mL of DMSO solution to obtain 1 mg/mL sample solution. Preparation of ergothioneine test solution: 10 mg of EGT was dissolved in 10 mL of distilled water to obtain 1 mg/mL sample solution. Preparation of tetrahydropiperine test solution: 10 mg of THP was dissolved in 10 mL of DMSO solution to obtain 1 mg/mL sample solution. Preparation of tetrahydrocurcumin+tetrahydropiperine test solution: 1 mg/mL THC solution and 1 mg/mL THP solution were mixed at 1:1. That is, put 1 mL of each solution into a 5 mL centrifuge tube, invert the tube repeatedly and shake it on a shaker to mix the test solution evenly. Preparation of ergothioneine+tetrahydropiperine test solution: 1 mg/mL EGT solution and 1 mg/mL THP solution were mixed at 1:1. That is, put 1 mL of each solution into a 5 mL centrifuge tube, invert the tube repeatedly and shake it on a shaker to mix the test solution evenly. Preparation of ergothioneine+tetrahydrocurcumin+tetrahydropiperine test solution: 1 mg/mL EGT solution, 1 mg/mL THC solution and 1 mg/mL THP solution were mixed at 1:1:1. That is, put 1 mL of each solution into a 5 mL centrifuge tube, invert the tube repeatedly and shake it on a shaker to mix the test solution evenly.

As shown in the table below, tyrosine solution and sample solution were added to a 96-well plate in turn, and incubated at 30° C. for 10 minutes. Then tyrosinase solution was added, mixed well, incubated at 30° C. for 20 min, and the absorbance was measured at 475 nm.

Group

| | Sample | Blank control | | |
|---|---|---|---|---|
| Reagent | group | 1 | 2 | 3 |
| Tyrosine (μL) | 180 | 180 | 180 | 180 |
| Sample (μL) | 10 | 10 | 0 | 0 |
| PBS (μL) | 0 | 0 | 10 | 10 |
| Mix well and incubate at 30° C. for 10 min | | | | |
| Tyrosinase (μL) | 10 | 0 | 10 | 0 |
| PBS (μL) | 0 | 10 | 0 | 10 |

Mix well and incubate at 30° C. for 20 min
Measure absorbance at 475 nm

The sample group was zeroed with blank control group 1, and the blank control group was zeroed with blank control group 3. The absorption value of the sample group is A, the absorption value of the blank control group 1 is A1, the absorption value of the blank control group 2 is A2, and the absorption value of the blank control group 3 is A3. The calculation formula of inhibition rate is as follows:

$$Inhibition rate = [ 1 - [ ( A - A 1 ) / ( A 2 - A 3 ) ] \times 100 \%$$

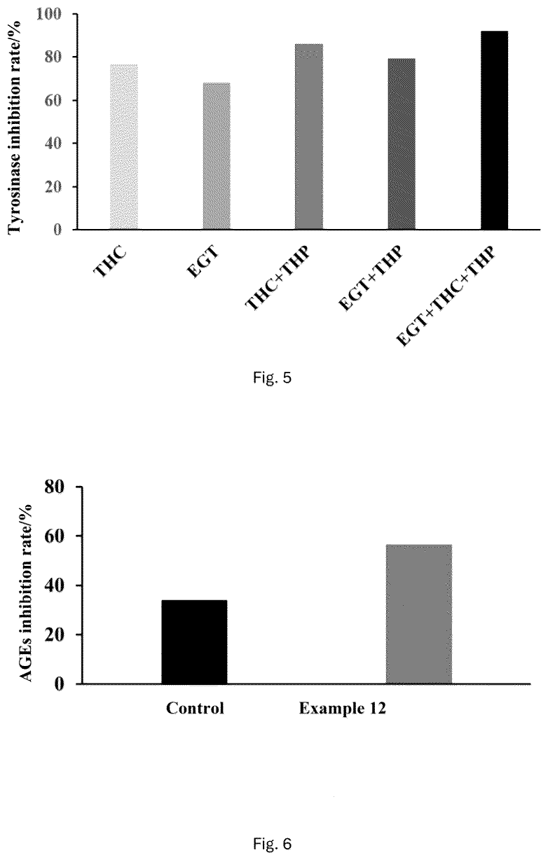

The results are shown in FIG. 5, which shows inhibitory effects of THC, EGT, THC+THP, EGT+THP and THC+EGT+THP on tyrosinase activity. As can be seen from FIG. 5, the inhibition rate of THC on tyrosinase is about 77%, the inhibition rate of EGT on tyrosinase is 68%, the inhibition rate of THC+THP on tyrosinase is 86%, the inhibition rate of EGT+THP on tyrosinase is 79%, and the inhibition rate of EGT+THC+THP on tyrosinase is 92%. THC shows excellent ability to inhibit tyrosinase activity, which is better than EGT. The combined effect of THC+EGT+THP is better than that of THC+THP and EGT+THP.

The results show that the inhibitory rate of the composition of the present invention on tyrosinase activity can reach about 80%, preferably above about 90%. Anti-glycation test: The inhibitory effect of the composition of the present invention on AGEs was tested.

Bovine serum albumin (BSA) and D-ribose were put together, and the formation of glycation products was observed in the presence of the compositions of Examples 1-14 of the present invention and the compositions of Comparative examples.

A bovine serum albumin solution with a concentration of 1 mg/mL was dissolved in phosphate buffered saline (PBS) and incubated with D-ribose with a concentration of 10 mM and each of the above compositions at 37° C. for 10 days.

As known, the final glycation end products exhibit light emission in the range of 420-600 nm after light excitation with a length of 300-420 nm. So, the glycation was evaluated by measuring the fluorescence of the AGEs at λem=450 nm emitted by each sample after excitation at λex=380 nm. The inhibition of glycation was visualized by the decrease in fluorescence compared with the samples treated with the sugar alone.

FIG. 6 shows the inhibition rate of the composition of Example 12 on AGEs. As can be seen from FIG. 6, the inhibition rate of AGEs in the control group is 33.8%, and the inhibition rate of the composition of Example 12 on AGEs is 56.4%. Therefore, the composition of Example 12 has a good inhibitory activity on the formation of fluorescent AGEs. The composition of the invention can inhibit AGEs by about 50%, preferably by more than about 55%.

Moisturizing test: The moisture retention effect of the composition of the present invention was tested.

### Preparation of a Topical Humectant Containing the Composition:

A humectant containing the compositions of Examples 1 to 14 of the present invention or the compositions of Comparative examples was prepared respectively. The humectant may also contain one or more of: hyaluronic acid, vitamin E, cannabidiol, EGCG/green tea, polyhydroxy acid, glycolic acid, superoxide dismutase, tea tree oil, psoralen, grape seed oil, vaseline or squalene or other suitable conventional humectant components.

### Clinical Efficacy Study:

Thirty healthy female subjects with mild to moderate photoaging and Fitzpatrick skin type I-II and 35-70 years old were recruited for clinical efficacy study. Consider inclusion criteria and exclusion criteria.

Subjects were randomly assigned into groups. The subjects were asked to each use a moisturizer containing the compositions of Examples 1-14 of the present invention on their right or left face, and to use a mild moisturizer or a moisturizer containing the compositions of Comparative examples on the opposite face, respectively. Neither the subjects nor the researchers knew the composition of the cream. The research coordinator developed and maintained blind research. Distribute diaries to the subjects to record the daily use of moisturizer, provide a return schedule, and request them to return to the research center in the 12th week. At each return (checkpoint), the enrolled subjects completed the baseline efficacy and tolerance assessment on both sides of the face. Skin elasticity and corneal measurement were measured and recorded from both sides of the face. In addition, at the 12th week (the end of the study), both sides of the face were wiped for microbial analysis. Subject diaries were checked for compliance and collected.

### Efficacy Evaluation:

Dermatologists or researchers evaluated efficacy parameters: fine lines, wrinkles, texture, gloss, brightness, smoothness, skin tone evenness (Evenness), compactness and pores. All assessments were conducted at baseline and at week 12 on a 5-point scale (0=none, 1=minimum, 2=mild, 3=moderate and 4=severe). Each side of the face was evaluated separately (the data was recorded in the table below).

### Wipe and Microbial Analysis:

At baseline and at week 12, both sides of the face were wiped for microbial analysis. Two swabs were generated for each wipe, with a total of 4 swabs for each subject. The swabs were stored in the refrigerator at −80° C. until the end of the study, and then transported (on dry ice) to the commercial laboratory in Vancouver, Canada for quantitative analysis of the microbiome.

Obtain facial swab according to the following instructions: Step 1: Immerse fresh swab in molecular water without DNA. Tighten the skin area with one hand. Hold the swab with the other hand so that the axis is parallel to the skin surface. Press hard, rub the swab back and forth 50 times (lasting 30 seconds), and slowly rotate the swab while wiping. Step 2: Carefully put the swab back into the test tube to ensure that the swab, swab stick and container are not touching any surface. Step 3: Put the test tube into the biological sample bag and freeze it at −80° C. until it is ready for shipment.

TABLE 1
Tolerance

Mann-Whitney

| rank and two- | Baseline | | | | Week 12 | | | |
|---|---|---|---|---|---|---|---|---|
| tailed pairing | Itching | Stinging | Burning | Irritation | Itching | Stinging | Burning | Irritation |
| Mean effective | 0.00 | 0.00 | 0.00 | 0.00 | 0.00 | 0.00 | 0.00 | 0.00 |
| value | | | | | | | | |
| Mean control | 0.00 | 0.00 | 0.00 | 0.00 | 0.00 | 0.00 | 0.00 | 0.07 |
| Effective value | 1.000 | 1.000 | 1.000 | 1.000 | 1.000 | 1.000 | 1.000 | 0.434 |
| v. Control | | | | | | | | |
| value p= | | | | | | | | |

TABLE 2
Efficacy evaluation

| | Baseline | | | Week 12 | | |
|---|---|---|---|---|---|---|
| | Mean | | Effective | Mean | | Effective |
| | effec- | | value v. | effec- | | value v. |
| | tive | Mean | Control | tive | Mean | Control |
| Score | value | control | value p= | value | control | value p= |
| fine lines | 2.58 | 2.57 | 0.791 | 2.54 | 2.54 | 1.000 |
| wrinkles | 2.54 | 2.57 | 0.791 | 2.54 | 2.54 | 1.000 |
| texture | 2.54 | 2.61 | 0.505 | 2.07 | 2.32 | 0.023 |
| gloss | 2.07 | 2.46 | 0.152 | 1.64 | 1.89 | 0.035 |
| brightness | 1.75 | 2.18 | 0.126 | 1.50 | 1.60 | 0.025 |
| smoothness | 1.93 | 2.07 | 0.471 | 1.54 | 1.75 | 0.214 |
| Eveness | 3.00 | 3.00 | 0.835 | 2.79 | 2.86 | 0.782 |
| compactness | 2.64 | 2.68 | 0.782 | 2.43 | 2.61 | 0.250 |
| pores | 2.56 | 2.57 | 0.791 | 2.50 | 2.54 | 0.792 |

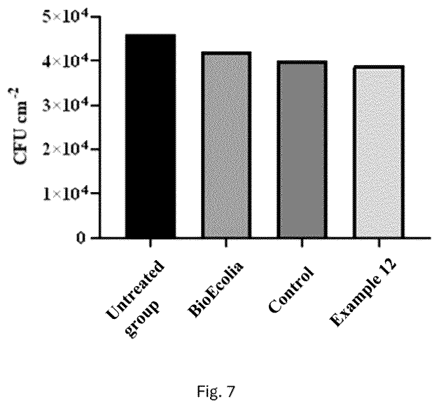

FIG. 7 shows the colonization of Propionibacterium acnes. As can be seen from FIG. 7, the colonization number of Propionibacterium acnes in the untreated group is 4.61×10⁴; BioEcolia is a special saprophytic substrate, and the colonization number of Propionibacterium acnes thereof is 4.20×10⁴; the colonization number of Propionibacterium acnes in solvent group (control group) is 4.01×10⁴, and that of the composition of Example 12 is 3.89×10⁴. It can be seen that the composition of Example 12 can reduce the colonization of Propionibacterium acnes.

Table 1 is a tolerance evaluation. As can be seen from Table 1, the composition of Example 12 was evaluated on each side of the subject's face, and there was no statistically significant tolerance problem. Table 2 is the efficacy evaluation. As can be seen from Table 2, after 12 weeks of applying the composition of Example 12, the skin texture, gloss and brightness of the subject's skin were improved statistically compared with the baseline.

The above are only the preferred embodiments of the present invention, and are not intended to limit the present invention. Those skilled in the art can make many changes, modifications, substitutions and variations on these embodiments without departing from the principles and purposes of the invention, and the scope of the invention is defined by the claims and their equivalents.

What is claimed is:

1. A skin care composition, comprising a first active component and a second auxiliary component, wherein the first active component is selected from one or more of tetrahydrocurcumin, ergothioneine, phloretin, arbutin, resveratrol, vitamin C, dipotassium glycyrrhizinate, ceramide, Aloe vera polysaccharide, Opuntia dillenii polysaccharide, Astragali radix polysaccharide, Dendrobii caulis polysaccharide, Cucumis metuliferus polysaccharide, thearubigins, retinol, carnosine; the second auxiliary component is selected from one or more of tetrahydropiperine, laurocapram, sucrose laurate, polysorbate, hydroxyethyl cellulose.

2. The skin care composition of claim 1, wherein the skin care is one or more of relieving photoaging of skin, reducing fine lines and wrinkles of skin, improving skin elasticity, improving skin compactness, improving skin gloss, brightness or texture, reducing or eliminating acne, relieving skin inflammation, keeping skin moist, improving or evening skin tone.

3. The skin care composition of claim 1, wherein the weight ratio of the first active component to the second auxiliary component is 1:20 to 50:1.

4. The skin care composition of claim 1, wherein the first active component is selected from one or more of tetrahydrocurcumin, ergothioneine.

5. The skin care composition of claim 1, wherein the second auxiliary component is selected from one or more of tetrahydropiperine, laurocapram, sucrose laurate.

6. The skin care composition of claim 1, wherein the second auxiliary component is selected from one or more of tetrahydropiperine, laurocapram.

7. The skin care composition of claim 1, wherein the first active component is tetrahydrocurcumin and/or ergothioneine, the second auxiliary component is tetrahydropiperine.

8. The skin care composition of claim 1, wherein the weight ratio of the first active component to the second auxiliary component is 1:2 to 5:1.

9. The skin care composition of claim 1, wherein the composition inhibits tyrosinase activity by about 80%.

10. The skin care composition of claim 1, wherein the composition inhibits TNF-α, IL-6 or IL-8 by more than 20% respectively.

11. The skin care composition of claim 1, wherein the composition inhibits AGEs by about 50%.

12. The skin care composition of claim 1, wherein the skin care composition is used for whitening, anti-inflammatory, anti-glycation, moisturizing or skin firming.
